(19) United States
(12) Patent Application Publication
(10) Pub. No.: US 2009/0299172 A1
(43) Pub. Date: Dec. 3, 2009

(54) Water Diffusion imaging and Uspio

(75) Inventors: Claire Corot, Lyon (FR); Marc Port, Deuil La Barre (FR); Philippe Robert, Paris (FR); Michel Schaefer, Lagny (FR); Denis Le Bihan, Saint-Nom-La-Breteche (FR); Johannes M. Fröhlich, Zollikon (CH)

Correspondence Address: BIRCH STEWART KOLASCH & BIRCH, PO BOX 747, FALLS CHURCH, VA, 22040-0747, US

(73) Assignee: GUERBET, VILLEPINTE (FR)

(21) Appl. No.: 12/308,089

(22) PCT Filed: Jun. 6, 2007
(86) PCT No.: PCT/EP2007/055598

§ 371 Date: Jun. 19, 2009

(30) Foreign Application Priority Data
Jun. 6, 2006 (FR) 0604989
Jan. 30, 2007 (FR) 0700661

(51) Int. Cl. A61B 5/055

(52) U.S. Cl. 600/420

(57) ABSTRACT

The invention relates to a method of water diffusion imaging in magnetic resonance imaging in an area of diagnostic interest, characterized in that it comprises, in combination, the administration of a contrast product capable of generating a signal specifically in its specific location area, said location area being included in said area of interest, the application of a water diffusion imaging sequence to the whole area of interest, and the reading of the images in the area of interest, the specific signal due to the contrast product significantly and specifically modifying the signal in the specific location area relative to the signal of the whole area of interest.

The invention relates to an imaging method comprising the combined use of a water diffusion imaging technique and contrast agents having an effect on the MRI signal at an area of diagnostic interest. The contrast agents are in particular superparamagnetic nanoparticles (USPIO).

Water diffusion imaging (or DWI, standing for diffusion weighted imaging) is already known as a way of diagnosing areas that exhibit a signal difference by this imaging between a healthy area and a pathological area, in particular a tumoral area. By using a suitable diffusion sequence, for example of spin echo type, on a tissue, there is measured, relative to a reference sequence not sensitive to diffusion, a reduction of the signal that is as great as the sequence is strongly weighted for the diffusion or the diffusion of the water in the tissue is rapid (exponential decrease). This loss of signal is often called “hyposignal”.

More specifically, the DWI method is a method for viewing the random movements of the molecules of water in the tissues, providing information on the mobility of the water molecules within biological tissues. In practice, at least two molecular motion probing gradient (MPG) pulses are applied either side of radiofrequency pulses at 180° in order to sensitize the signal to the diffusion. The duration and the intensity of the MPG pulses is represented by the value b (in s/mm²). The contrast is typically obtained using a weighted imaging T2 acquired by rapid imaging sequences such as EPI (echo planar imaging) and/or parallel imaging methods such as SENSE (sensitivity encoding) in the brain. In the rest of the body, improvements have also been obtained with the use of HASTE, FASE (single shot fast echo), SSFP and other techniques, and the increase in excitations (NEX) for a better signal/noise ratio. For example, a slice thickness of 5 to 10 mm with an NEX value of approximately 2 is used. To study more extensive areas for which a higher resolution is required, a slice thickness of 5 mm or less with an NEX value of 5 to 10 is used. Complementary viewing methods are also used, such as MIP, MPR, VR. Known imaging parameters are detailed, for example, in Takahara et al, Radiation Medicine, vol. 22, No. 4, 275282, 2004, page 276.

In a tumoral area, the increased cellularity (the number of cells by volume) is accompanied by a reduced diffusion of the water molecules. For a given diffusion time, the diffusion distance is reduced. The tumoral area therefore appears as a “relative hypersignal” relative to the reference tissue.

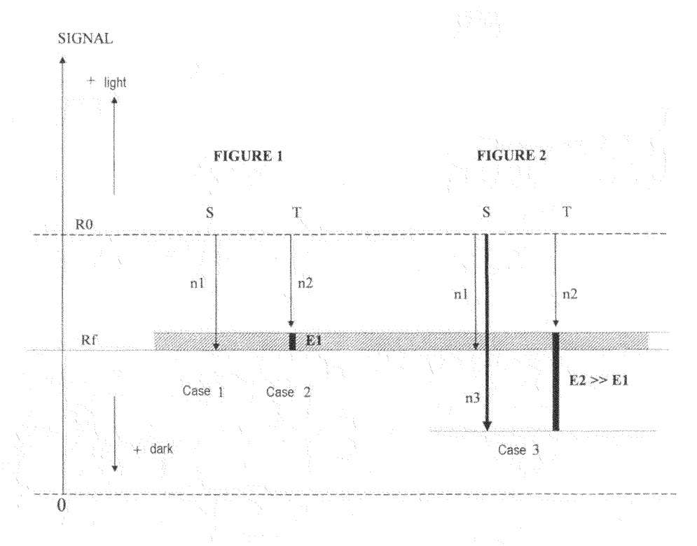

A signal difference is thus measured between a healthy area and a tumoral area, corresponding to the case of FIG. 1:
Case 1: in the healthy tissue (S), the diffusion signal (corresponding to a normal apparent diffusion coefficient ADC) is lowered by a quantity n1 to a level Rf relative to the reference level R0: this is a “hyposignal”
Case 2: in the tumoral tissue (T), the diffusion signal (corresponding to a lower apparent diffusion coefficient ADC) is lowered less (n2<n1, associated with a lower diffusion of the water). It is also said that, by diffusion imaging, the signal reduces less in a tumoral area than in a healthy area: the tumour therefore appears as a relative hypersignal (lighter) compared to the healthy tissue.

There is thus obtained a contrast image showing the difference E1=(n1−n2).

In practice, to study lymph nodes by diffusion imaging T2 for example, there is thus observed in principle a strong hyposignal n1 in a healthy node and a moderate hyposignal n2 in a metastatic node: in the images, the healthy node appears dark and the pathological node light (hypersignal from the metastatic node).

A variant of diffusion imaging is a diffusion imaging designated DWIBS (DWI background suppression), intended more particularly for viewing areas of interest of large dimensions, and in particular the whole body, in regions where certain tissues have a low diffusion coefficient (fats in particular). This imaging, designed to improve DWI imaging, makes it possible to eliminate the signal corresponding to these tissues, in particular by eliminating the signal emitted by the fats, and is advantageous, for example, for imaging cancerous metastases. The DWI imaging described above has in fact limitations in certain cases: (i) the molecular diffusion imaging method is sensitive to the motions of the organs produced by breathing which limits the time of the imaging session and thus the thickness and the number of excitations, (ii) the suppression of the signal from the fats can be insufficient despite the use of IR-SE-EPI (inversion-recovery spin echo EPI) and CHESS (chemical shift) imaging, above all for a 3D imaging: the residual hypersignal from the fat at the periphery or inside certain organs is superimposed on the internal areas and can conceal or mimic pathological tissues.

A DWIBS technique that uses STIR-EPI (short T1 inversion recovery) is in particular described in Radiation Medicine, vol. 22, No. 4, 275-282, 2004 for example with a 1.5 Tesla scanner, with a slice thickness of 4 to 9 mm and a value b of 500 to 1000 S/mm².

In addition to the suppression of the background signal, the DWIBS applies an inversion of the B&W scale or of the colours to obtain an image similar to that obtained in PET (positron emission tomography) imaging: the pathological tissues (for example metastatic nodes) appear in black, and the healthy tissues (for example the healthy nodes) in white. The principle is represented in FIG. 3, bearing in mind that, in practice, a DWIBS imaging normally comprises the following steps:
preparation of the magnetization by suppressing the signal from the fats
application of the diffusion sequence: the healthy tissues appear dark and the pathological tissues white (as for DWI described above)
filtering and transformation by the software of this image in order to obtain an image similar to that obtained in PET imaging: the pathological tissues appear darker (signal N′1, inverse of the signal n1 of Case 1 in FIG. 1) than the healthy tissues U.S. Pat. No. (signal N′2, inverse of the signal n2 of Case 2 in FIG. 1).

However, such DWI or DWIBS diffusion imaging techniques are not always specific enough to diagnose a pathological, and in particular cancerous, area. Such is the case in particular when the cellularity is not necessarily accompanied by a cancerous pathological condition or risk, hence difficulties in distinguishing, for example, a malignant tumour from a tumour also with reduced diffusion but benign. Such is the case also when the organs being studied have a high cellularity even in the healthy state. The contrast (difference E1 in FIG. 1 of FIGS. 1 and 3) is then not sufficient for a totally reliable diagnosis.

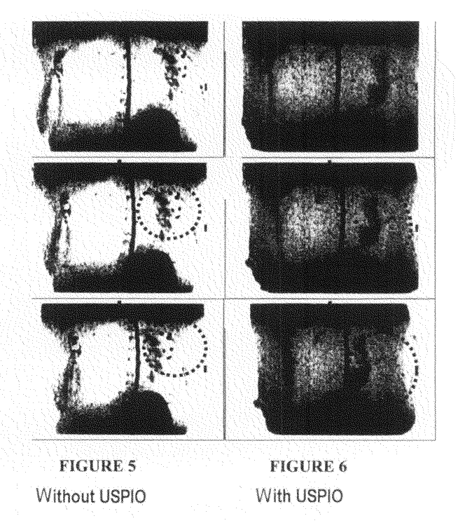

Physically, by taking the example of sentinel nodes in DWIBS imaging, corresponding to the case of Photo 1 in FIG. 5:
the fats (suppressed) are white (or very light grey)
the spinal column appears very light grey and the spinal cord appears black
the spleen appears black
all the nodes appear lightly dark (dark grey), without it being possible to confirm here that they are healthy or benign nodes or tumoral nodes (in typical cases, tumoral nodes appear clearly in black).

There is therefore still a need for a more sensitive and more specific imaging making it possible to confirm the non-pathological state and/or better characterize the pathological state, in particular tumoral, and notably the metastases.

To resolve these technical problems, the applicant has used, in combination with water diffusion imaging, contrast agents, in particular superparamagnetic particles, and more especially iron oxides commonly designated USPIO (ultrasmall superparamagnetic iron oxide). These contrast agents are particularly suited to MRI imaging of T2/T2* type. This combined use in one and the same imaging of the patient of contrast agent leads to a synergy effect between these two techniques. This same imaging can proceed, where appropriate, in a number of phases, particularly if the action time of the contrast product requires an injection of the product prior to the imaging to allow the contrast agent to reach the pathological or healthy region.

More specifically, the contrast agents can, because of their local magnetic field gradients, bring about a modification of the signal in the area in which they come to be located specifically (designated specific location area or ZLS in this application).

This synergy is reflected in the emphasis of the signal difference between a healthy and pathological area when the healthy area has a different (higher) diffusion coefficient than the pathological area and picks up the contrast agent (USPIO with T2* effect) differently from (more than) the pathological area. The signal in the healthy part is therefore doubly lowered, by the effect of the diffusion and by that of the contrast agent. As indicated in FIG. 2, in the case of DWI imaging (corresponding to the case of FIG. 1, without DWIBS type inversion), the contrast agent is specifically located in the healthy area and causes therein a signal drop in T2 imaging. Compared to FIG. 1 (without contrast product), the injected contrast product that comes to be located specifically in the healthy tissue (S) generates therein an additional signal drop. The resultant is a hyposignal signal n3 in the healthy tissue greater than the hyposignal n1, so a greater contrast is obtained (difference E2=n3−n2). The hyposignal in the healthy area will then be more pronounced (and the tumoral area will appear with a more pronounced relative hypersignal) than with the use of the diffusion technique alone or of the contrast agent alone.

In the image, the healthy areas appear significantly darker than the benign areas, which makes it possible specifically to better identify the tumoral areas.

In the case of DWIBS imaging, or a similar method with signal inversion, the injected contrast product which comes to be located specifically in the healthy tissue (S) generates therein an additional signal rise. Thus, a healthy node that appeared dark in DWIBS imaging without contrast product (and could possibly be difficult to distinguish from a tumoral node) has, thanks to the injection of the product, its signal significantly increased (it becomes substantially lighter), whereas the tumoral node whose signal is not modified remains dark. Only the tumoral nodes remain apparent and dark.

Compared to FIG. 3, the signal N′3 (inverse of the signal n3) is significantly greater than the signal N′2 (inverse of the signal n2). FIG. 6 illustrates the case of a patient for which all the nodes become light (and therefore disappear): they are therefore healthy, non-tumoral, nodes. It can also be seen that the spleen becomes much lighter because the USPIO is located in a known manner also in this elimination organ. There is thus obtained a synergy between diffusion and suppression of the healthy tissues, thanks to the elimination of substantially all the signals from the healthy tissues by the combined effect of the suppression of fat, of the suppression of water (diffusion), and of the contrast product distinctive of the benign lesions.

The invention thus relates, according to one aspect, to the use of contrast agent in a method of water diffusion imaging in magnetic resonance imaging in an area of diagnostic interest, comprising in combination:
a) the administration of a contrast product capable of generating a signal specifically in its specific location area, said location area being included in said area of interest
b) the application of a water diffusion imaging sequence to the whole area of interest
c) the reading of the images in the area of interest, the specific signal due to the contrast product significantly and specifically modifying the signal in the specific location area relative to the signal of the whole area of interest.

In the case of the USPIOs, the step a) typically precedes the step b).

The invention relates, according to an embodiment, to a method of diagnostic imaging by MRI comprising the application of a water diffusion sequence being accompanied by a hyposignal in an area of diagnostic interest, the diffusion imaging supplying a hyposignal of strong diffusion (p) in a part of the area of interest having a strong water diffusion (in particular, an area with low cellularity), and a hyposignal of moderate diffusion (q<p) in a part of the area of interest having a low diffusion (in particular, an area with strong cellularity, an area with increased density of the extracellular matrix), the method also comprising the administration of a contrast product capable of reaching specifically the area with low cellularity and generating a signal modifying the diffusion signal only and specifically in the area with low cellularity. Broadly, this concept is applied to factors capable of varying the diffusion of the water molecules in the pathological area linked to various mechanisms such as hypercellularity, the increase in density of the extracellular matrix, the cellular swelling due to an oedema.

This technique is particularly advantageous in the case of a DWIBS imaging of the whole body to diagnose certain cancers such as prostate cancer. Use will advantageously be made of USPIOs such as Sinerem® to display the healthy nodes (the product recognizes specifically the healthy nodes that include macrophages).

In order to obtain the described synergy, the contrast product and the methods of administration and the imaging parameters of this product will be chosen in such a way that the effect of the product on the signal is quantifiable during the diffusion imaging that is carried out (DWI or DWIBS, or similar). In other words, the diffusion signal modification assigned to the contrast product takes place in the measurement window of the diffusion imaging method used. The contrast product can thus be injected at different moments depending on the nature of this product and the time it needs to generate the signal specific to it.

For example, a product is injected on a day D, this product generating its signal on the day D+1. On the day D+1, the measurement of the diffusion signal (20-minute DWI or DWIBS imaging sequence, for example) and the measurement of the signal from the contrast product are performed simultaneously, these two signals then being aggregated for the diagnosis.

The imaging parameters will advantageously be as follows: b between 100 and 1500, preferably between 500 and 1000 sec/mm², TR (repetition time)1500-5000 ms, TE (echo time) 50-80 ms, TI (inversion time) 150-180 ms, NEX (number of excitations) 2-10, slice thickness 2-20 mm.

The combination of diffusion and contrast agent thus offers a two-fold advantage: improved specificity (healthy/pathological distinction), and sensitivity (better sensitivity than diffusion imaging alone, which makes it possible to detect a smaller tumour in particular).

The diffusion imaging will help to better characterize pathological areas, to follow the physiopathological trend to obtain a more precise functional imaging, in particular the tumoral progression stage. It will also help to identify effective treatments, to monitor the effectiveness of a therapeutic treatment in the areas in which the contrast agent used will be used, and therefore in particular of the treatments used in therapy (medicines and candidate medicines).

The invention also relates to a method of diagnostic imaging by MRI comprising the application of a water diffusion sequence being accompanied by a signal in an area of diagnostic interest with strong cellularity that is not distinctive between a healthy part and a pathological part of this area, also comprising the administration of a contrast product capable of generating an additional signal distinctive between the healthy part and the pathological part.

The invention also relates to the use of a USPIO for any imaging method described in the present application, and the use of a USPIO for the preparation of a diagnostic composition that can be used in any imaging method described in the present application. More broadly, these results show all the benefit of combining a given imaging technique with the use of contrast products providing additional functional information and allowing for a better sensitivity and/or specificity of the diagnosis. For the imaging according to the invention, depending on the embodiments, the contrast product is advantageously a superparamagnetic product, in particular a nanoparticle of iron oxide covered with a polysaccharide or a carbohydrate. Use will advantageously be made of USPIOs, in particular of particles covered with a coating of polysaccharide type chosen from dextrans or derivatives, inasmuch as they have the effect explained above on the diffusion of the water molecules. The dextran derivatives can contain at least one acid group, or several functional groups comprising atoms O, N, S, P. Carboxy or polycarboxydextrans can in particular be used. It is also possible to use as coating, coatings described in Chemical Reviews, 2004, vol. 104, No. 9, 3893-3946, cited in particular in Tables 9 to 12, and in particular those covering iron oxides.

The superparamagnetic particles that can be used are advantageously very small particles of ferrite, including in particular magnetite (Fe₃O₄), maghemite (y-Fe₂O₃) and other magnetic mineral compounds of transition elements, of a size less than approximately 100-150 nm.

According to an embodiment, carboxylic derivatives of polysaccharides such as starch or carboxydextran and carboxylalkyldextran derivatives (reduced or not reduced), such as carboxymethylic, carboxyethylic, carboxypropylic. This coating of the magnetic particles is intended to obtain a stability of the colloidal solutions of magnetic particles, also called ferrofluids, in a physiological medium. The syntheses that make it possible to obtain these types of particles are known, for example described in Robert S. Molday and D. Mackenzie; J. of Immunological Methods (1982), 52, pp. 353-367) or Chem. Commun. 2003, 927-937. Such covered particles are described, for example, in the documents EP 656 368, WO 98/05430, EP 450 092. Among the particles that can be used, covered with a coating of polymeric or non-polymeric derivatives, there are: Sinerem® (Combidex®), ferrumoxitol, SHU 555A (Resovist®, carboxydextran-based coating, described in particular in Radiology, 2001, vol. 221, 237-243), SHU555C (Supravist®), NC100150 (starch coating described in particular in Magn. Res. Mat. in Physics, Biology and Medicine, 1999, 8: 207-213), VSOP (citrate-based coating described in particular in Prog. Colloid Polym. Sci., 1996, 100: 212-216, and Journ. Magn. Res. Imag., 2000, 12: 905-911, EP 888 545), particles of MION and CLIO type, ADMSs, such as the refined derivatives of these various compounds still at the pre-clinical stage. Also worthy of note are the nanoparticles described in WO2006012201, WO2006/031190, US2005/0260137, WO2004/107368, WO2006/023888. Also worthy of mention are nanoparticles of iron oxide covered with a phosphonate or derivative coating, and in particular gem-bisphosphonate, described in WO 2004/258475 and in particular in the formula of Example 16 (aminoalcohol chain type coverage).

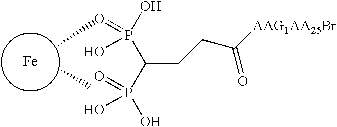

It is also possible to use as the coating, macromolecules such as proteins like albumin or synthesis polymers such as methacrylates and organosilanes, galactanes [Josephson L., Groman E. V., Menz E. et al; Magnetic Resonance Imaging 8; 616-637; 1990], starch [Fahlvik A. K., Holtz E., Schroder U. et al; Invest. Radiol. 25; 793-797; 1990], glycosaminoglycanes [Pfefferer D, Schimpfky C., Lawaczeck R.; SMRM-Book of abstracts 773; 1993]. It is also possible to use as coating PEG and aminoalcohol branches.

The hydrodynamic diameter of the basic structure of the USPIO/SPIOs used in solution is typically between 2 and 500 nm, preferably 2 to 50 nm. The relaxivities r1 and r2 of a magnetic contrast product give the measure of its magnetic effectiveness and make it possible to assess its influence on the recorded signal. The relaxivity r1 of the particles that can be used in the context of the present invention is advantageously of the order of 10 to 50 mMol-1s-1 and their relaxivity r2 of the order of 20 to 400 mMol-1s-1, at 20 MHz. The iron content of the particle (% by weight) is of the order of 20 to 60%, typically 30 to 50%.

The USPIO/SPIOs are typically used with a dose of 0.1 mol/kg to 10 mmol/kg of metal, preferably of 1 mmol/kg to 5 mmol/kg, by injection or perfusion in an artery or a vein. The individual doses will depend on the composition of the magnetic particles, on the administration pathway, on the type of diagnosis required, and on the patient.

The USPIO/SPIOs are typically in the form of stable colloidal solutions (or suspensions of stabilized particles) and can be formulated in the form of lyophilized powders to be associated with an appropriate solvent. Their administration pathway is known to those skilled in the art, typically intravenous, but also by local application (mammary carcinoma for example). The compositions are preferably administered by parenteral pathway, by oral pathway, other administration pathways not, however, being excluded, the administration in the form of an intravenous injection being particularly preferred. When administration by oral pathway is envisaged, the compositions of the invention are, for example, in the form of capsules, effervescent tablets, bare or coated tablets, sachets, sugar-coated tablets, ampoules or oral solutions, microgranules or forms with prolonged or controlled release. Products for oral administration are known such as Lumirem®.

Any contrast agent of the prior art can be tested in appropriate conditions to determine good conditions of use in diffusion imaging, and by using an imaging in T1 and/or T2 and/or T2* mode. It is possible in particular to use complex contrast products of paramagnetic metal ions such as gadolinium (in particular any chelate chosen from the following and their derivatives known to those skilled in the art: DTPA, DOTA, DO3A, HPDO3A, PCTA, MCTA, BOPTA, DOTMA, AAZTA, TETA, PDTA, gadofluorines, TRITA), hyperpolarized agents, shift agents (cest).

The invention applies to diagnostic indications for which the diffusion imaging alone gains by being coupled with the use of contrast products. The following diagnostic indications are particularly worthy of note: oncological imaging (liver, lungs, breasts, etc.), imaging of the pelvis, whole-body or territory-by-territory check-up, check up for adenopathies, lymphomas, metastases, melanomas, imaging for characterizing healthy tissues.

According to the embodiments, diffusion imaging with contrast product is combined with the administration of a therapeutic product, so as to measure the effectiveness or to diagnostically monitor a therapeutic treatment (in particular, to monitor chemotherapy, hormone therapy, for example for the prostate). According to advantageous embodiments, the use of the following for inflammatory areas is avoided (so as not to suppress their signal): atheromatous plaque, multiple sclerosis, degenerative disorders. From the exemplary embodiments described, it will be understood that various combinations can be produced in DWI or DWIBS diffusion imaging or their possible refinements (such as diffusion sequences designated off-resonance saturation) so as to obtain a distinctive and specific signal thanks to the contrast agent. These various cases are advantageously used according to the diagnostic indication concerned, the ability of the contrast product to lower or raise the signal in T1 and/or T2/T2* imaging mode, and to specifically target the healthy area or the tumoral area.

The invention is illustrated by figures:

FIG. 1: schematic diagram of DWI imaging without contrast agent

FIG. 2: schematic diagram of DWI imaging with contrast agent

FIG. 3: schematic diagram of DWI imaging with signal inversion (DWIBS or similar imaging) without contrast agent

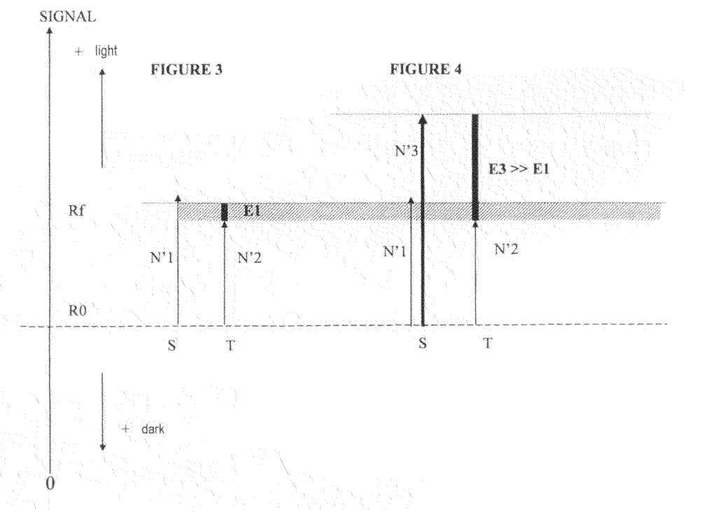

FIG. 4: schematic diagram of DWIBS imaging with contrast agent

FIG. 5: photo of a patient without injection of contrast agent

FIG. 6: photo of a patient after injection of contrast agent

### EXAMPLE

### Case of a USPIO (Sinerem®) to Study Healthy/Metastatic Sentinel Nodes

The Sinerem® is administered at T0 to a human with a dose for example of 1.1 to 3.4 mg Fe/kg, in particular 2.6 mg Fe/kg. The imaging is performed after 24 to 36 hours with an appliance of 1.0 to 3.0 Tesla (Philips Achieva, Best, the Netherlands), in particular 1.5 Tesla.

The parameters are as follows:

Spools freely chosen (depending on the place being. viewed): for the body, “SENSE BODY”
Field of view: 400
Rectangular field of view: 70%

### Matrix: 160×256

Scan percentage: 80%
VOXEL size: 1.5×1.5×4 mm³
SENSE if checking a part of the body like the neck: in AP direction, factor 2 (for the whole body, without SENSE factor).
Layers: 60 (3 to 4 times)
Slice thickness: 4.00 mm
“Foldover” direction: anterior-posterior
Scan mode: multislice 2D
Inversion sequence: 180 ms at 1.5 T
Fast imaging mode: echo planar imaging
Echo time: 70 ms
Repetition time: “shortest” mode (depending on the number of layers)
“half scan”: mode “yes” with a factor of 0.6
“Water-fat shift”: minimum mode
“Diffusion mode-sequence”: spin echo
“b-factors”: 0 and 1000

The diffusion sequence applied is a DWIBS sequence (“diffusion weighted whole body imaging with background body signal suppression”): “single-shot IR-EPI diffusion weighted imaging”. The MIP (“maximum intensity projection”) projections of the images b=800 to 1000 are inverted and reconstructed.

The imaging session is performed at T0 (before administration) to recognize the lymph nodes and after 24 to 36 or 48 hours to improve the characterization (“staging”). The healthy lymph nodes disappear, related to the susceptibility effect associated with EPI sequences.

Compared to PET imaging of the whole body, this technique which makes it possible to reconstruct 2D layers for the whole body leads to a better spatial resolution.

Thus, these results allow for a particularly advantageous improvement of the diagnosis when the use of Sinerem® (Combidex® or other nanoparticle) is not totally satisfactory to guarantee a distinction between healthy tissue (node) and pathological tissue.

The USPIO+diffusion combination is applied to indications that are very different between themselves and possibly complementary:
analysis of a part of a tissue, distinguishing therein healthy and pathological areas,
tissue characterization, of a tissue relative to another tissue, making it possible to perfectly distinguish individualized tissues (some being healthy, the others being pathological): in this case, the diagnostic benefit is significant for the diagnostic indication concerned, by making it possible to steer, for example, towards a selective antitumoral treatment or a surgical procedure to remove pathological nodes (unlike a tissue part analysis for which the selective treatment or removal are not always possible at the level of only a part of the tissue).
imaging and characterization of territories that are not accessible to certain contrast agents but accessible to others, for example in the case of deep territories (in particular inflammatory, circulatory, nervous systems).

1. Method of water diffusion imaging in magnetic resonance imaging in an area of diagnostic interest, characterized in that it comprises, in combination:
a) the administration of a contrast product capable of generating a signal specifically in its specific location area, said location area being included in said area of interest
b) the application of a water diffusion imaging sequence to the whole area of interest
c) the reading of the images in the area of interest, the specific signal due to the contrast product significantly and specifically modifying the signal in the specific location area relative to the signal of the whole area of interest.

2. Method according to claim 1, in which the signal measured from the representative values of the water diffusion in the area of interest is a hyposignal.

3. Method according to claim 2, in which the hyposignal is associated with an apparent diffusion coefficient (ADC), related to the cell density and/or viscosity.

4. Diagnostic imaging method by MRI according to claim 2, comprising the application of a water diffusion sequence being accompanied by a hyposignal in an area of diagnostic interest, the diffusion imaging supplying a strong diffusion hyposignal in a part of the area of interest that has a low cellularity, and a moderate diffusion hyposignal in a part of the area of interest having a high cellularity, wherein it also comprises the administration of a contrast product capable of reaching specifically the area with low cellularity and generating a signal modifying the diffusion signal only and specifically in the area with low cellularity.

5. Diagnostic imaging method by MRI according to claim 1, comprising the application of a water diffusion sequence being accompanied by a signal in an area of diagnostic interest with strong cellularity that is not distinctive between a healthy part and a pathological part of this area, wherein it also comprises the administration of a contrast product capable of generating an additional signal distinctive between the healthy part and the pathological part.

6. Method according to claims 1, 4 or 5, in which the contrast product is a superparamagnetic product.

7. Method according to claim 6, in which the superparamagnetic product is a nonoparticle of iron oxide covered with dextran or starch, or any derivative of dextran, where appropriate with polyethylene glycol derivative groups grafted on.

8. Method according to claims 1, 4 or 5, in which the diffusion imaging sequence is a DWI or DWIBS imaging sequence, or their variants.

9. Method according to claims 1, 4 or 5, wherein it is combined with the administration of a therapeutic product, so as to measure the effectiveness or handle the diagnostic follow-up of a therapeutic treatment.

10. The method according to claim 6 in which the contrast product is USPIO.

11. (canceled)

12. The method according to claim 10, for a whole-body or territory-by-territory check-up, check-up for adenopathies, lymphomas, metastases, melanomas.

13. The method according to claim 10 for the study of deep territories.

14. The method according to claim 10 for the characterization of healthy tissues.

15. The method according to claim 6 wherein the superparamagnetic product is a nanoparticle of iron oxide covered with a polysaccharide or a carbohydrate or a phosphonate or bisphosphonate group.

16. The method according to claim 7, wherein the derivative of dextran is carboxydextran, carboxyalkyl dextran.

17. The method according to claim 14 for the characterization of sentinel nodes.
